(19) United States
(12) Patent Application Publication
(10) Pub. No.: US 2002/0182217 A1
(43) Pub. Date: Dec. 5, 2002

(54) Time release chelators

(76) Inventor: Bruce Halstead, Grand Terrace, CA (US)

Correspondence Address: Rutan & Tucker LLP, Suite 706, 611 Anton Blvd., Costa Mesa, CA, 92626, US

(21) Appl. No.: 10/159,434

(22) Filed: May 29, 2002

Related U.S. Application Data
(60) Provisional application No. 60/294,478, filed on May 30, 2001.

(51) Int. Cl. A61M 36/14; A61K 51/00; A61K 39/42; A61K 33/14; A01N 59/10

(52) U.S. Cl. 424/159.1; 424/1.61; 424/675

(57) ABSTRACT

A pharmacological composition comprises a chelator in a time release formulation in a quantity sufficient to reduce a serum concentration of a bivalent metal in an amount of at least 20% for a period of at least 8 hrs. Preferred chelators chelate at least one of Ca²⁺ and Mg²⁺.

This application claims the benefit of U.S. provisional application No. 60/294,478 filed May 30, 2001, incorporated herein by reference in its entirety.

### FIELD OF THE INVENTION

The field of the invention is antiviral compositions.

### BACKGROUND OF THE INVENTION

Numerous antiviral drugs are known in the art, however, all or almost all of them suffer from one or more disadvantages. Particularly problematic in the administration is of such drugs is their relatively low solubility and/or comparably short serum half-life time. Consequently, many patients need to follow a strict regimen to maintain effective serum concentration of such drugs, frequently resulting in repeated disruptions of an otherwise productive lifestyle. Therefore, there is a need for improved antiviral compositions that are well tolerated, simple to administer, and maintain a relatively long serum half-life.

### DETAILED DESCRIPTION

The inventors contemplate that treatment of a viral infection can be significantly improved by administration of a chelator in a time-release formulation. Furthermore, the inventors contemplate that the chelator is co-administered in a time-release formulation with a second or further agent with antiviral effect (which may be administered following a conventional protocol or in a second time release formulation). Contemplated viruses include retroviruses (e.g., HIV, HCV), ssDNA and dsDNA.

Consequently, the inventors contemplate a pharmacological composition that includes an a chelator in a time release formulation in a concentration such that a single administration of the chelator reduces the serum concentration of a bivalent metal in an amount of at least 20% for a period of at least 8 hours, more preferably at least 30% for a period of at least 10 hours, and most preferably at least 40% for a period of at least 12 hours.

With respect to the chelator it is generally contemplated that all chelating agents are suitable for use in conjunction with the teachings presented herein so long as such chelators (a) reduce serum concentration of a bivalent metal (e.g., Ca²⁺ and Mg²⁺) in an amount of at least 25%, and (b) are at least partially effective to promote viral disintegration at an administered dosage. Particularly contemplated bivalent metals include Ca²⁺ and Mg²⁺. Particularly preferred chelators include 1,2-Bis(2-aminophenoxy)ethane-N,N,N′,N′-tetraacetic acid, Ethylenebis(oxyethylenenitrilo)tetraacetic acid, 1,2-bis(2-aminophenoxy)ethane-N,N,N′,N′-tetraacetic acid tetrakis(acetoxymethyl ester), trans-1,2-diaminocyclohexane-tetraacetic acid, and diethyllenetriamine-pentaacetic acid, tri-methylaminetricarboxylic acid, poly(aspartic acid), and poly(glutamic acid), ethylenediamine-N,N,N′,N′-tetraacetic acid, and EGTA.

There are numerous methods of preparing a time release formulation known in the art, all of which are contemplated suitable for use in conjunction with the teachings herein. However, particularly contemplated time release formulations include ion exchange resins, encapsulations with acid or base resistant coatings, compacting the formulation to control solvation, slow-melting carriers, enzyme-degradable carriers, etc.

Depending on the amount chelator in contemplated compositions, it is contemplated that the viral titer in the serum of a patient infected with the virus will decrease at least 10% for at least 4 hours, more preferably at least 25% for at least 6 hours, and most preferably at least 40% for at least 8 hours after administration of a single dose of contemplated compounds.

It should further be appreciated that contemplated compositions may further comprise direct antiviral agents and/or indirect antiviral agents. As used herein, the term “direct antiviral agent” refers to an agent that directly interferes with one or more viral components. For example, virus protein specific antibodies, reverse transcriptase inhibitors or protease inhibitors are considered direct antiviral agents, because such compounds directly bind and to and/or reduce the activity of their respective viral target structures. As also used herein, the term “indirect antiviral agent” refers to a compound that indirectly interferes with a replication or propagation of a virus, and particularly include immunomodulatory agents (e.g., cytokines, various nucleoside analogs, and/or Zn²⁺). However, it should be appreciated that chelators are explicitly excluded from the definitions of direct and indirect antiviral compounds.

With respect to the administration of contemplated compositions, it should be recognized that various protocols are suitable, and especially contemplated protocols include oral and parenteral administration (e.g., via a tablet, syrup, injection, suppository, topical administration transcutaneous administration, etc.). Consequently, the dosage and formulation of contemplated compositions may vary substantially. However, it is generally preferred that a single dosage is within the range of about 10 mg to about 3000 mg.

Thus, specific embodiments and applications of chelators in time release format have been disclosed. It should be apparent, however, to those skilled in the art that many more modifications besides those already described are possible without departing from the inventive concepts herein. The inventive subject matter, therefore, is not to be restricted except in the spirit of the appended contemplated claims. Moreover, in interpreting both the specification and the contemplated claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

What is claimed is:

1. A pharmacological composition, comprising:
a chelator in a time release formulation in a quantity sufficient to reduce a serum concentration of a bivalent metal in an amount of at least 20% for a period of at least 8 hrs.

2. The composition of claim 1 wherein the chelator is in a quantity sufficient to reduce a serum concentration of a bivalent metal in an amount of at least 30% for a period of at least 10 hrs.

3. The composition of claim 1 wherein the chelator is in a quantity sufficient to reduce a serum concentration of a bivalent metal in an amount of at least 40% for a period of at least 12 hrs.

4. The composition of claim 1 wherein the chelator chelates at least one of Ca²⁺ and Mg²⁺.

5. The composition of claim 4 wherein the chelator is selected from the group consisting of 1,2-Bis(2-aminophenoxy)ethane-N,N,N′,N′-tetraacetic acid, Ethylenebis(oxyethylenenitrilo)tetraacetic acid, 1,2-bis(2-aminophenoxy)ethane-N,N,N′,N′-tetraacetic acid tetrakis(acetoxymethyl ester), trans-1,2-diaminocyclohexane-tetraacetic acid, and diethyllenetriamine-pentaacetic acid.

6. The composition of claim 4 wherein the chelator is selected from the group consisting of tri-methylaminetricarboxylic acid, poly(aspartic acid), and poly(glutamic acid).

7. The composition of claim 4 wherein the chelator is ethylenediamine-N,N,N′,N′-tetraacetic acid.

8. The composition of claim 1 wherein the bivalent metal is at least one of Ca²⁺ and Mg²⁺.

9. The composition of claim 1 further comprising an antiviral agent.

10. The composition of claim 9 wherein the antiviral agent is selected from the group consisting of a cytokine, an antibody, Zn²⁺, a reverse transcriptase inhibitor, a protease inhibitor, and an antibody.

11. The composition of claim 1 wherein the composition reduces a viral serum titer of a virus in an amount of at least 10% for a period of at least 4 hours.

12. The composition of claim 1 wherein the composition reduces a viral serum titer of a virus in an amount of at least 25% for a period of at least 6 hours.

13. The composition of claim 1 wherein the composition reduces a viral serum titer of a virus in an amount of at least 40% for a period of at least 8 hours.

14. The composition of claim 10 wherein the virus is a retrovirus.

15. The composition of claim 14 wherein the retrovirus is an HIV virus or an HCV virus.
